(19) United States
(12) Patent Application Publication
(10) Pub. No.: US 2012/0093717 A1
(43) Pub. Date: Apr. 19, 2012

(54) MICROSPHERE/NANOFIBER COMPOSITES FOR DELIVERY OF DRUGS, GROWTH FACTORS, AND OTHER AGENTS

(75) Inventors: Robert Mauck, Philadelphia, PA (US); Lara Ionescu, Philadelphia, PA (US); Jason Alan Burdick, Philadelphia, PA (US); Brendon Baker, Philadelphia, PA (US)

(73) Assignee: The Trustees of the University of Pennsylvania, Philadelphia, PA (US)

(21) Appl. No.: 13/148,965

(22) PCT Filed: Jan. 29, 2010
(86) PCT No.: PCT/US10/22573

§ 371 Date: Nov. 8, 2011

Related U.S. Application Data
(63) Continuation of application No. 61/154,366, filed on Feb. 21, 2009.

(51) Int. Cl. A61K 9/14; A61K 38/02; B29C 53/14; A61K 35/00; B29C 47/00; A61K 51/12; A61K 38/39

(52) U.S. Cl. 424/1.11; 424/400; 514/1.1; 514/17.2; 424/93.1; 264/465; 264/171.13

(57) ABSTRACT

Provided are compositions that include polymeric fibers and microspheres entrapped within the fibers, the compositions being capable of controlled delivery of one or more agents while also maintaining their structural properties. Also provided are related methods of fabricating these compositions and methods of utilizing the compositions to deliver agents to a subject.

### RELATED APPLICATIONS

The present application claims the benefit of U.S. Application No. 61/154,366, filed on Feb. 21, 2009, the entirety of which is incorporated herein by reference for all purposes.

### TECHNICAL FIELD

The present invention relates to the fields of biodegradable polymer compositions and to the field of controlled release of drugs or other agents.

### BACKGROUND

Fibrous tissues are often characterized by a dense, ordered collagenous structure that defines their unique and anisotropic mechanical properties. These properties are critical for tissue function, and are compromised in instances of injury and tissue degeneration. Fibrous tissues are also known for their poor intrinsic healing capacity.

Injuries to fibrous tissues are common. For example, it is estimates that there about 70 meniscus (knee) tears per 100,000 persons each year, and there are more than 250,000 knee replacements performed in the United States each year.

Some efforts have been directed toward the fabrication of scaffold structures that are also capable of delivering drugs or other active agents. These existing scaffolds, however, are of limited utility because the active agents are incorporated directly into the material of the scaffold such that drug delivery can only be accomplished by degradation of the scaffold.

More specifically, such structures that degrade concurrent with drug delivery are suboptimal because such structures are of limited use to patients whose conditions or injuries require the physical support of the supportive scaffold before, during, and after drug delivery. Accordingly, there is a need in the art for implantable, supportive drug delivery systems where the system's ability to delivery a drug or other agent is decoupled from the structure's mechanical properties, i.e., where the structure is capable of providing support during and after drug delivery. The value of such systems would be further enhanced if the systems were capable of supporting cell growth and proliferation that

### SUMMARY

In meeting the described challenges, the present invention first provides engineered fibrous compositions, comprising: one or more first fibers comprising a first polymeric material, the first polymeric material having a first rate of degradation when contacted with an fluid medium; one or more second fibers comprising a second polymeric material, the second polymeric material having a second rate of degradation when contacted with an fluid medium, the second rate of degradation being faster than the first rate of degradation; and one or more microspheres, the one or more microspheres having a third rate of degradation when contacted with a fluid medium.

Also provided are methods of fabricating engineered fibrous compositions, comprising: forming one or more first fibers from a first solution comprising a first polymer, the first solution comprising one or more microspheres, the first fibers having a first rate of degradation when contacted with a fluid medium, and the microspheres having a second rate of degradation when contacted with a fluid medium; and forming one or more second fibers from a second solution comprising a second polymer, the second fibers having a second rate of degradation when contacted with a fluid medium, and the second rate of degradation being faster than the first rate of degradation.

The present invention also provides engineered fibrous compositions, comprising: one or more first fibers comprising a first polymeric material, the first polymeric material having a first rate of degradation when contacted with an fluid medium; one or more microspheres disposed adjacent to one or more first fibers, the one or more microspheres having a second rate of degradation when contacted with a fluid medium.

Also disclosed are methods of delivering an agent to a subject, comprising: disposing within the subject an engineered fibrous composition according to the claimed invention so as to give rise to at least a portion of the engineered fibrous composition being contacted with a fluid medium.

Further provided are methods of delivering an agent, comprising, contacting a composition with a fluid medium, the composition comprising one or more first fibers comprising a first polymeric material, he first polymeric material having a first rate of degradation when contacted with the fluid medium, ne or more second fibers comprising a second polymeric material, the second polymeric material having a second rate of degradation when contacted with an fluid medium, the second fibers degrading faster than the first fibers when contacted with the fluid medium, and one or more microspheres disposed among the first and second fibers, the one or more microspheres degrading more slowly than the second fibers when contacted with the fluid medium, and the one or more microspheres being capable of releasing one or more agents when contacted with the fluid medium; the contacting being performed so as to at least partially degrading one or more second fibers, the contacting being performed such that one or more microspheres remains disposed among at least the first fibers, and the contacting being performed such that one or more microspheres releases one or more agents into the environment exterior to the microsphere.

### BRIEF DESCRIPTION OF THE DRAWINGS

The summary, as well as the following detailed description, is further understood when read in conjunction with the appended drawings. For the purpose of illustrating the invention, there are shown in the drawings exemplary embodiments of the invention; however, the invention is not limited to the specific methods, compositions, and devices disclosed. In addition, the drawings are not necessarily drawn to scale. In the drawings:

FIG. 1 illustrates a bead situated at surface of a PCL scaffold made according to the claimed invention (PEO originally present in the scaffold was already removed);

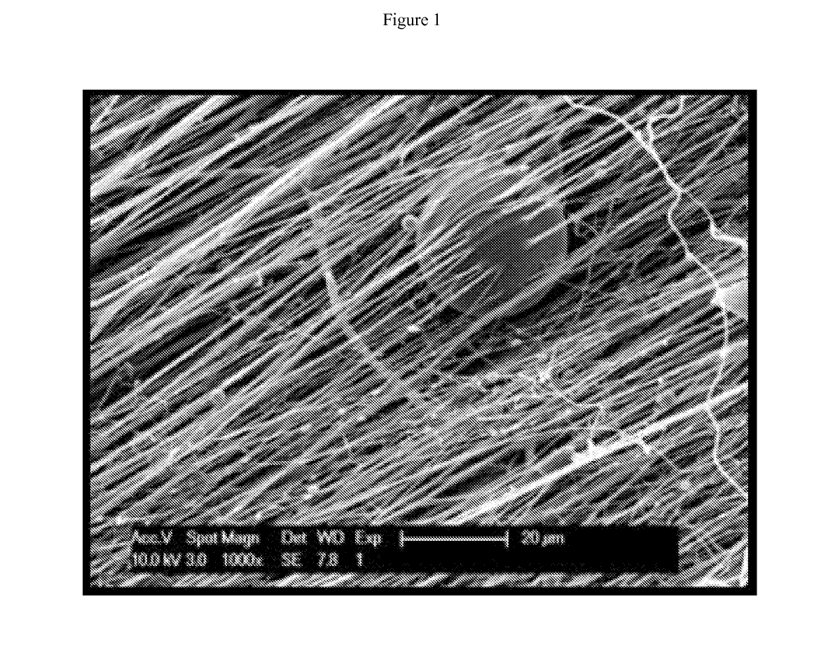

FIG. 2 illustrates a plurality of beads disposed within a scaffold made according to the present invention;

FIG. 3 illustrates a plurality of beads disposed within a PCL scaffold according to the present invention (PEO has already been removed) and illustrates the depth to which beads or microspheres may be disposed within an inventive scaffold;

FIG. 4 illustrates several beads or microspheres disposed within a scaffold according to the present invention where the scaffold has been cut—the edge of the cut illustrates the density of fibers in this exemplary scaffold;

FIG. 5 illustrates fluorescent microspheres disposed within a network of PEO nanofibers (scale bar=50 microns);

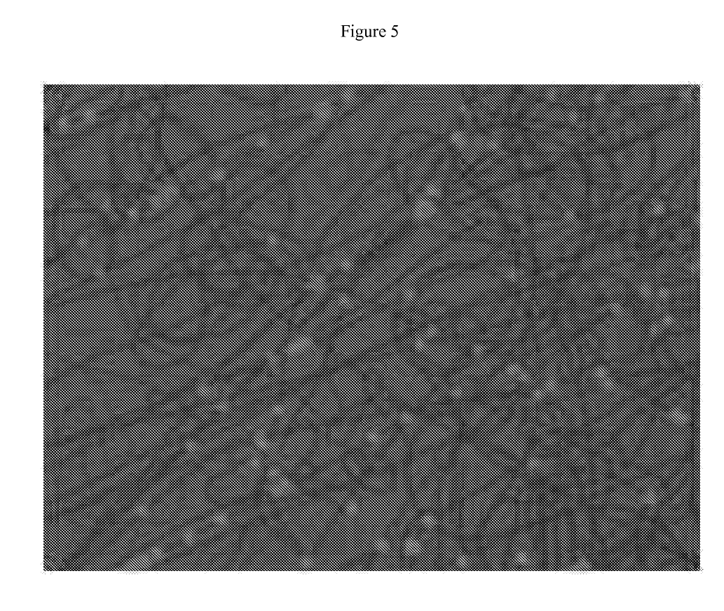

FIG. 6 illustrates a SEM of microspheres disposed within aligned PEO nanofibers (scale=2 microns);

FIG. 7 illustrates (A) fluorescent microspheres within PEO nanofibers (scale: 50 μm), micrographs (B) and quantification (C) of microspheres in PEO nanofibers with increasing bead density in spinning solution (ROI: region of interest, Scale: 50 μm), (D) SEM of microspheres within aligned PEO nanofibers (scale: 2 μm). * p<0.05 vs lower concentration.

FIG. 8 illustrates (A) dual electrospun PCL and PEO/microspheres fibers. (B) After aqueous incubation, PEO fibers dissolve, leaving microspheres entrapped in the remaining PCL network (scale: 10 microns);

FIG. 9 illustrates a light micrograph (A, scale: 50 m) and SEM (B, scale: 20 microns) of fabricated microspheres, (C) BSA release from PEO scaffolds with increasing mass of microspheres in spinning solution;

FIG. 10 illustrates fabrication of drug-delivering nanofibrous scaffolds—microspheres delivered through sacrificial PEO fibers are entrapped within the PCL fibrous network after PEO removal;

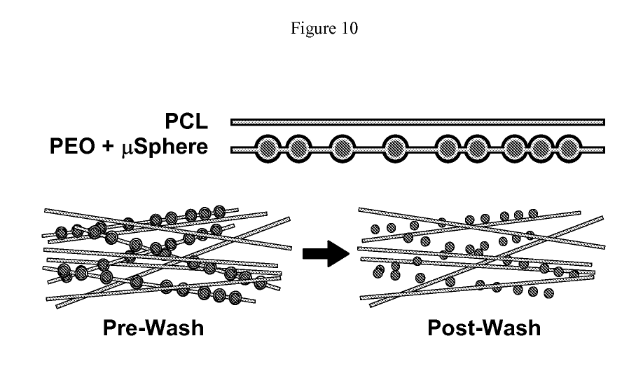

FIG. 11 illustrates bright-field images of PEO fiber mats formed from solutions with differing MS density, scale=500 μm, MS areal density in PEO mats as a function of MS density in spinning solutions (data represent the average of 4 measurements from 3 independent spinning solutions, and *indicates significant difference from lower values);

FIG. 12 illustrates PCL/MS scaffolds (after PEO removal) with increasing MS concentration in the spinning solution-inset: quantification of BSA delivery per gram of scaffold (n=3, scale 200 μm);

FIG. 13 illustrates mechanical properties of aligned scaffolds with increasing MS density (*indicates significant difference from control (no MS), p<0.05);

FIG. 14 illustrates (A) schematic of modified collection mandrel for direct electrospinning of meniscus implants, (B) native sheep meniscus histology (collagen: lighter gray area on left-hand side of crescent-shaped region proteoglycan: darker gray area on right-hand side of crescent-shaped region) and schematic of localized growth factor delivery from entrapped microspheres;

FIG. 15 illustrates the fabrication of microsphere-laden nanofibrous scaffolds, where (A) shows composite light and fluorescent micrograph showing electrospun PCL fibers with embedded PS microspheres (diameter 2 microns) distributed along the fiber length (Scale bar=50 μm), and (B) shows SEM micrograph demonstating alterations in PCL fiber morphology local to the inclusion of an 15.7 micron diameter PS microsphere (Scale bar=25 μm);

FIG. 16 illustrates the dose-dependent inclusion of PLGA microspheres in nanofibrous mats, wherein (A) shows SEM micrograph showing PLGA microspheres fabricated by the double emulsion technique (Scale bar=50 μm), (B) shows a histogram of microsphere diameter at after fabrication, filtering, and washing, (C) shows PLGA microsphere density with a field of view (FOV) of a PEO fiber mat increases with increasing microsphere density in the electrospinning solution. *indicates significant difference compared with lower values, p<0.05, and (D) shows bright-field images of PEO fiber mats formed from solutions of increasing PLGA MS density (Scale bar=500 μm);

FIG. 17 illustrates a non-limiting approach for decoupling drug delivery from scaffold mechanics, wherein composite scaffolds are formed from microspheres delivered through a sacrificial PEO fiber fraction coupled with a stable PCL fiber fraction (Pre-Wash), and with dissolution of the PEO (After-Wash), MS remain entrapped within the slow degrading and surrounding fibrous PCL fibrous network;

FIG. 18 illustrates the realization of composite MS-laden scaffolds with sacrificial content, showing bright-field with overlaid fluorescent image (A, 4×, Scale bar=50 μm) and SEM (B, Scale bar=20 μm) of PEO/PCL/MS composite—in (A), bright dots show PLGA MS, PCL fibers and sacrificial PEO fibers are also labeled within the composite structure—after PEO removal, microspheres remain entrapped and distributed between the remaining PCL fibers (C and D, arrows, Scale bar=10 μm);

FIG. 19 illustrates the construction and mechanical analysis of composite MS-laden scaffolds, wherein (A) shows a schematic of electrospinning PCL/PCL-MS scaffold, (B) shows that stiffness of scaffold decreases with increasing MS density (Control=0, Low=0.05, Med=0.1, High=0.2 g MS/mL electrospinning solution), (C) shows that modulus decreases with increasing MS density, (D) shows a schematic of electrospinning PCL/PEO-MS scaffold, (E) shows that stiffness does not change with increasing MS density, and (F) shows that modulus decreases at medium and high density MS inclusion, but not at low inclusion density (*indicates p<0.05 from control); and

FIG. 20 illustrates the controlled release from composite MS-laden scaffolds, wherein (A) shows SEM of degraded free microspheres after 35 days in physiologic conditions (Scale bar=10 μm), (B) shows SEM of partially degraded microsphere in nanofibrous composite after 25 days in physiologic conditions (Scale bar=10 μm), (C) shows overlay of light and fluorescent micrographs showing mixed MS population (BSA MS and CS MS; scale bar=250 μm), (D) shows sustained release of bovine serum albumin (BSA) or chondroitin sulfate (CS) from PLGA microspheres with time in physiologic conditions, (E) shows sustained release of BSA and CS from composite PCL/PEO-MS scaffold containing either BSA or CS microspheres, and (F) shows sustained release of both BSA and CS from a single composite system containing both BSA and CS microspheres at a 1:1 ratio.

### DETAILED DESCRIPTION OF ILLUSTRATIVE EMBODIMENTS

The present invention may be understood more readily by reference to the following detailed description taken in connection with the accompanying figures and examples, which form a part of this disclosure. It is to be understood that this invention is not limited to the specific devices, methods, applications, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

It is to be appreciated that certain features of the invention which are, for clarity, described herein in the context of separate embodiments, may also be provided in combination in a single embodiment. Conversely, various features of the invention that are, for brevity, described in the context of a single embodiment, may also be provided separately or in any subcombination. Further, reference to values stated in ranges include each and every value within that range.

The present invention first provides engineered fibrous compositions. The inventive compositions suitably include one or more first fibers comprising a first polymeric material, the first polymeric material having a first rate of degradation when contacted with an fluid medium.

The compositions also include one or more second fibers comprising a second polymeric material, the second polymeric material having a second rate of degradation when contacted with an fluid medium, and the second rate of degradation being faster than the first rate of degradation.

One or more microspheres—or other structures capable of controlled degradation and release/elution of agents—are also suitably present in the compositions. The microspheres suitably have a third rate of degradation when contacted with a fluid medium.

In some embodiments, the second fiber degrades essentially instantaneously upon contact with an aqueous medium. In other embodiments, the second fiber degrades more slowly; degradation may take place over a period of seconds, minutes, hours, days, weeks, or even longer.

The microspheres—or other degrading structures—suitably contain one or more agents. Agents include active agents, labels, and the like. Fluorescent dyes, proteins, drugs, analgesics, growth factors, enzymes, and the like may be disposed within a microsphere, as can be vitamins, pharmaceuticals, and or any combination thereof. Labels—such as fluorescent labels, magnetic labels, radioactive labels, and the like may also be disposed within—and released from—the microspheres.

Microspheres are suitably biodegradable, and may include poly(lactic-co-glycolic acid), polyanhydride, or both. The degradation rate of the microspheres is suitably slower than the second rate of degradation. In some embodiments, the microsphere degrades or elutes one or more agents during degradation of the second fibers, after degradation of the second fibers, or both.

The first polymeric material is suitably biocompatible and comprises a polyester, a polyurethane, a protein, or any combination thereof. In some embodiments, the first polymeric material is rigid or capable of supporting adjacent structures. The first polymeric material suitably has a comparatively slow degradation rate; as explained elsewhere herein, it is preferable that the first polymeric material remain in place and provide some structural support (1) during (or even after) degradation of the microspheres (and, likewise, during the microspheres' elution or release of one or more agents) and (2) during or even after the degradation or dissolution of the second (or other) polymeric materials. Poly(caprolactone) is considered a particularly suitable first polymeric material, as are other biocompatible polymers that have comparatively slow degradation rates when exposed to internal environments. Proteins—such as silk—are also suitable first polymeric materials.

Polymers that degrade rapidly—by comparison—are suitable second polymeric materials. Polyesters, poly(ethylene oxide), proteins, and the like are all suitable second polymeric materials; poly-β-amino esters are considered especially suitable, as is collagen. For example, PCL-PEO fiber compositions are suitable, as are PCL-collagen fiber compositions; collagen can be fixed with genipin.

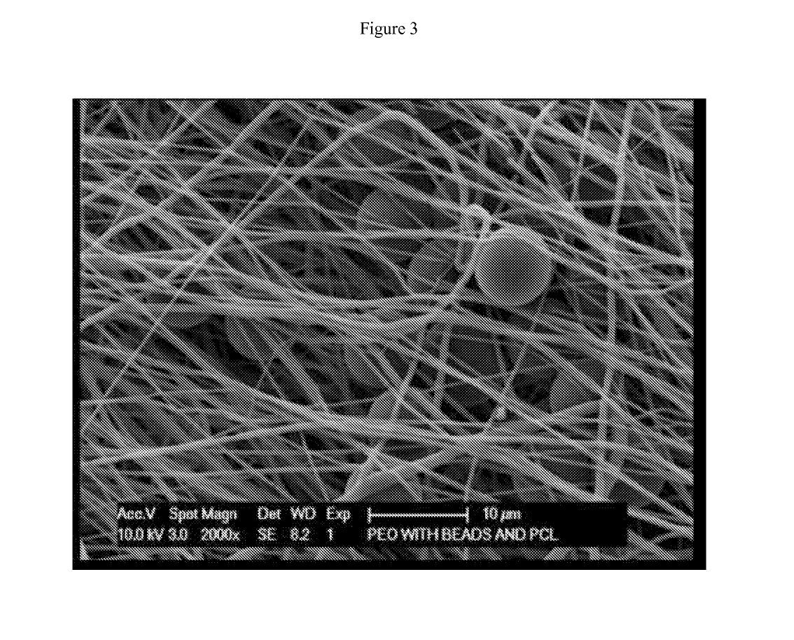

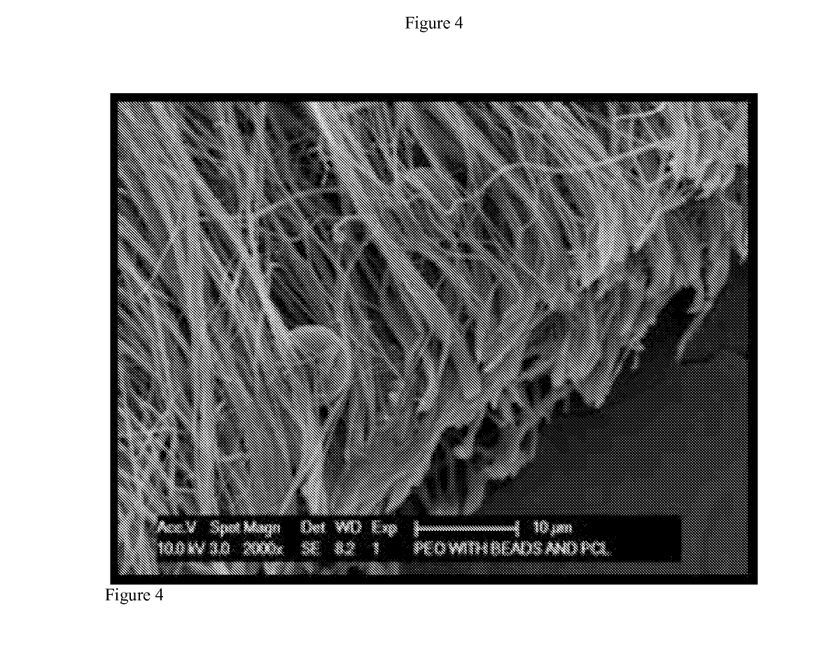

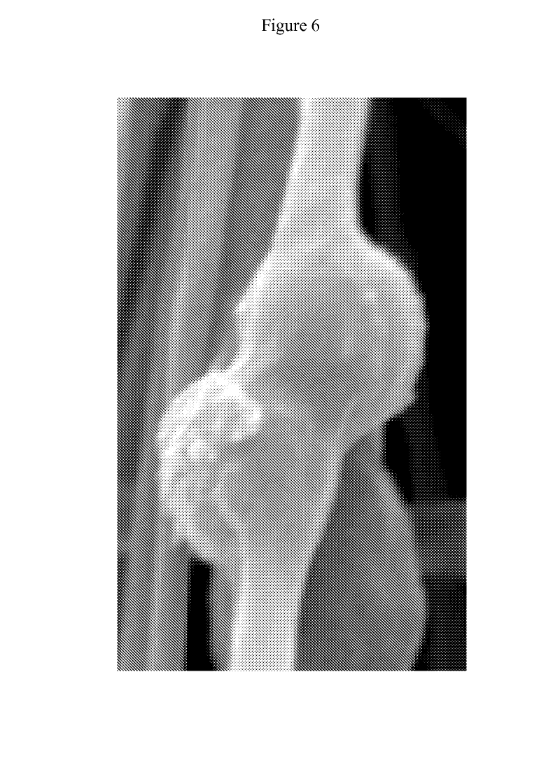

In some embodiments, one or microspheres resides at least partially within a second fiber, as shown in FIG. 6. In other embodiments, one or microspheres resides adjacent to a first fiber, a second fiber, or both, as shown in, e.g., FIGS. 3 and 4.

In some embodiments, one or more one or more first fibers are suitably intertwined with one or more second fibers. Without being bound to any particular theory of operation, this provides for microspheres to be entrapped by fibers that remain after the degradation of the fibers within which the microspheres partially resided. Also without being bound to any particular theory of operation, it is believed that the enhanced porosity that results from erosion of one fiber component of a scaffold (leaving behind the second fiber) enhances the ability of cells to infiltrate into the scaffold so as to proliferate and grow within the scaffold.

Biological materials may also be included in the claimed compositions. Collagen, cells, and other tissues may be disposed within the claimed compositions.

As shown in the figures, the fibers of the claimed invention may be of various dimensions. A fiber suitably comprises a cross-sectional dimension of from about 1 to about 10,000 nm; fibers of between about 200 nm to about 5,000 nm in diameter are considered especially suitable. In embodiments that include microspheres, the microspheres are suitably from about 0.01 up to about 40 or even 100 microns in diameter, although spheres or other delivery structures can have cross-sectional dimensions in the range of tens or hundreds of nanometers, depending on the needs of the user and on other design considerations. It is to be understood that although certain disclosed embodiments describe the use of microspheres, the invention contemplates the use of any suitable delivery vehicle—such as polymer “cages” or other structures—that is capable of containing and then releasing an agent under suitable, physiological conditions.

Because tissue regeneration often requires organized proliferation of cells, the inventive compositions suitably include two or more fibers that are aligned—longitudinally—with one another. In this way, cells or other tissues that may infiltrate the scaffolds or reside on the scaffold's surface will be provided an aligned structure upon which they can propagate in an aligned fashion such that the resultant tissue or structure achieves the desired mechanical properties.

To achieve the desired results, the user may vary the concentration of microspheres or delivery structures in the compositions. The compositions may include two or more kinds of microspheres or delivery structures so as to achieve release of two or more agents. The compositions may be constructed so as to have a gradient of microsphere concentration within such that the compositions exhibits a particular, tunable release profile for the agent or agents disposed within. The compositions may also include multiple polymeric materials so as to produce a composition having a specific profile of mechanical properties, which profile may include mechanical properties that change (e.g., the composition becomes less rigid) over time and with exposure to particular media. The release profile of a given composition may also be affected by the spatial distribution (and density) of microspheres and the degradation kinetics of the microspheres.

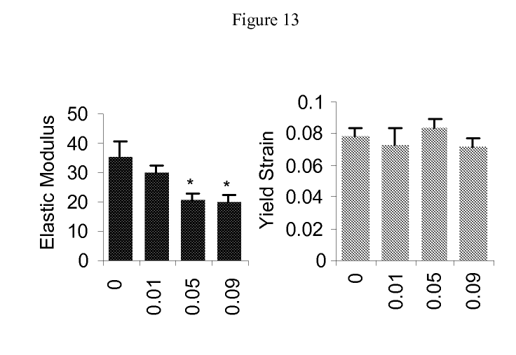

Because the release of agents disposed within the composition is independent of (i.e., de-coupled from) the mechanical properties of the composition, the user has the ability to tune both the mechanical properties and the agent release characteristics of the composition; as shown in FIG. 13, some mechanical properties of the scaffolds were unaffected by increasing microsphere concentration. Put another way, the inventive compositions provide so-called “sustained patterns” (in the form of polymeric fibers that do not immediately degrade upon exposure to physiological conditions) on which a subject's cells may grow and proliferate while the composition also releases agents into the subject to, e.g., promote healing and repair.

In some embodiments, the release of agents from the microspheres is strongly related to the microspheres' degradation. In others, the release of agents from the microspheres is less strongly-related to the microspheres' degradation, and relates to diffusion or some other property. It is preferred—though not required—that the microspheres be capable of releasing one or more agents before, during, and after (or during, or during and after) degradation of the second polymeric material and while the first polymeric material remains in place.

For example, in a PCL-PEO-microsphere scaffold, the PEO dissolves soon after the scaffold is implanted in a subject, leaving behind the microspheres entrapped by fibers of the comparatively long-lasting PCL. The microspheres then degrade so as to release one or more agents (or otherwise elute such agents) while the PCL fibers remain, thus resulting in a system capable of physically supporting adjacent tissues while at the same time releasing agents—such as growth factors—that are useful in treating a subject's condition. The scaffolds may be formulated such that one or more polymeric materials remains in place for days or even weeks; in other embodiments, the polymeric materials are chosen and formulated so as to achieve faster degradation of the scaffold. The optimal degradation profile will be dictated by the user's needs and will be easily achieved by manipulation of the polymeric materials and of process parameters, such as fiber thickness (which is controlled by, e.g., electrospinning process parameters) and the overall density of fibers.

The present invention also provides methods of fabricating an engineered fibrous composition. These methods include forming one or more first fibers from a first solution comprising a first polymer, the first solution comprising one or more microspheres, the first fibers having a first rate of degradation when contacted with a fluid medium, and the microspheres having a third rate of degradation when contacted with a fluid medium; and forming one or more second fibers from a second solution comprising a second polymer, the second fibers having a second rate of degradation when contacted with a fluid medium, the second rate of degradation suitably being slower than the first rate of degradation. In this way, the microsphere-carrying first fibers degrade before the fibers made of the second solution, thus leaving behind microspheres entrapped within the network of second fibers.

Formation of the fibers is suitably accomplished by electrospinning, which technique is well-known in the art. Electrospinning is an easily-tuned process, and the user of ordinary skill will encounter little difficulty in adapting the process to producing fibers of the desired dimensions and characteristics. The electrospinning may be accomplished by devices having one or more than one nozzles or jets; in this way, compositions that include two or more kinds of fibers with one or more kinds of microspheres or othe delivery devices can be easily formed. Rotating nozzles and multiple nozzles can be used to achieve fiber organization within scaffolds and meshes.

As described elsewhere herein, the solution that includes the first polymer may include one or more microspheres or other agent-delivery compositions. Suitable polymers and microspheres are described elsewhere herein. It is preferable—but not necessary—that the microspheres be essentially inert to the first solution.

The fibers may be of a cross-sectional dimension of from about 1 to about 10,000 nm; fibers having a cross-sectional dimension of from about 200 to about 5,000 nm are considered especially suitable.

The fabrication suitably includes intertwining one or more first fibers with one or more second fibers. In this way, the microspheres are entrapped within the fiber network. The fibers are also, in some embodiments, formed such that first and second fibers are aligned—longitudinal alignment is preferable—with one another. Scaffolds of non-aligned fibers are also within the scope of the claimed invention.

Also provided are engineered fibrous compositions, the compositions suitably including one or more first fibers comprising a first polymeric material, the first polymeric material having a first rate of degradation when contacted with an fluid medium; one or more microspheres disposed adjacent to one or more first fibers, the one or more microspheres having a second rate of degradation when contacted with a fluid medium.

Suitable polymeric materials and suitable microspheres are described elsewhere herein. In preferred embodiments, one or more microspheres are or even entrapped disposed between two or more first fibers. It is preferred that the microspheres be disposed within the fibers such that the microspheres remain within the composition when the composition is exposed to physiological conditions within a subject. One or more microspheres may, in some embodiments, be bound or otherwise secured to one or more first fibers.

It is preferred that the microspheres release agents disposed within while the first fibers remain in place so as to provide compositions capable of providing mechanical support while also releasing agents disposed within. To this end, suitable embodiments include microspheres that degrade more slowly than do the first polymeric materials. Put another way, it is preferable that the second rate of degradation be slower than the first rate of degradation.

As described elsewhere herein, it is preferred that two or more fibers be aligned longitudinally with one another.

Further disclosed are methods of delivering an agent to a subject. These methods include disposing within a subject an engineered fibrous composition according to the present invention so as to give rise to at least a portion of the engineered fibrous composition being contacted with a fluid medium, and the composition releasing one or more agents into the subject. In some embodiments, the methods are performed by placing the engineered fibrous composition adjacent to a tendon, a ligament, a meniscus, cartilage, an annulus fibrosus, cardiac tissue, vascular tissue, neural tissue, and the like. At least a portion of the engineered fibrous composition may be secured to the tendon, a ligament, a meniscus, cartilage, an annulus fibrosus, cardiac tissue, vascular tissue, neural tissue, or any combination thereof.

The compositions of the claimed invention may also include other agents disposed on the surface of or within the compositions. These agents may enhance or discourage cell adhesion to the composition. The compositions may also include colorants or other materials to aid visualization of the compositions.

The present invention also provides are methods of delivering an agent. These methods include contacting a composition with a fluid medium, where the composition includes (1) one or more first fibers comprising a first polymeric material, he first polymeric material having a first rate of degradation when contacted with the fluid medium, (2) one or more second fibers comprising a second polymeric material, the second polymeric material having a second rate of degradation when contacted with an fluid medium, the second fibers degrading faster than the first fibers when contacted with the fluid medium, and (3) one or more microspheres disposed among the first and second fibers, the one or more microspheres degrading more slowly than the second fibers when contacted with the fluid medium, and the one or more microspheres being capable of releasing one or more agents when contacted with the fluid medium.

The contacting of the composition with the fluid medium suitably at least partially degrades one or more second fibers such, the contacting being performed such that one or more microspheres remains disposed among at least the first fibers, and the contacting being performed such that one or more microspheres releases one or more agents into the environment exterior to the microsphere.

As a non-limiting illustration of these methods, a network composition of aligned PCL and PEO fibers with drug-eluting microspheres disposed within the fibrous composition is placed into a human or other animal subject. Upon contact with the physiological environment within the subject, the PEO fibers degrade, leaving behind the microspheres entrapped within the PCL fiber network. The microspheres degrade so as to release one or more agents disposed within (or, in some embodiments, the microspheres elute the agent or agents) while the PCL fibers remain in place, the method thus providing a method of delivering an agent to a location within a subject while also providing physical/structural support to the subject at that same location. Depending on the needs of the user, the composition may be formulated so as to provide a scaffold for cell growth, and may provide growth factors or other agents suitable for promoting, directing, or otherwise effecting and controlling such growth.

In some embodiments, the first (rigid) fibers degrade more slowly than do the second (sacrificial) fibers or the microspheres, and the microspheres degrade more slowly than do the second (sacrificial) fibers. In some embodiments, the first fibers are constructed so as to remain in place when subjected to a physiological environment for days, weeks, or longer, depending on the needs of the user. The microspheres may degrade at the same or at a different rate than the first, left-behind fiber. In some embodiments, the microspheres may include two or more agents so as to release different agents at different times or to release two or more agents simultaneously. In some embodiments, two or more kinds of microspheres are used so as to achieve release of the same or different kinds of agents at the same or at different times. The microspheres may include gradients of agents so as to release different amounts of an agent—or agents—at different times.

In addition, the fibers themselves may also include agents such that the fibers themselves also serve as a source of agent release. These agents may be chosen to supplement, complement, or otherwise reinforce agents that the microspheres may release.

In another embodiment, one type of fiber within the multi-fiber network is selectively etched away so as to leave behind microspheres (or other agent-delivery vehicles or compositions) disposed, entrapped, or otherwise remaining within the network of fibers left behind by removal of the first kind of fiber. Suitable polymers, microspheres, and drug delivery compositions are described elsewhere herein. Block copolymers and the like are considered suitable polymers for the claimed invention; it is preferable that the polymers, microspheres, and other components used in the invention are biocompatible.

One aspect of the claimed invention is its use in treating damaged tissues and other structures, such as reconstitution of the load bearing role of the knee meniscus via fabrication of implantable constructs. Such strategies would ideally recapitulate not only the micro- and nano-scale topography of the tissue ECM, but also the macro-scale anatomic form, which issues the present invention addresses by way of direct electrospinning of an entire meniscus implant. To enable this fabrication process, a rotating collecting mandrel is modified to form an annular wedge shaped crevice.

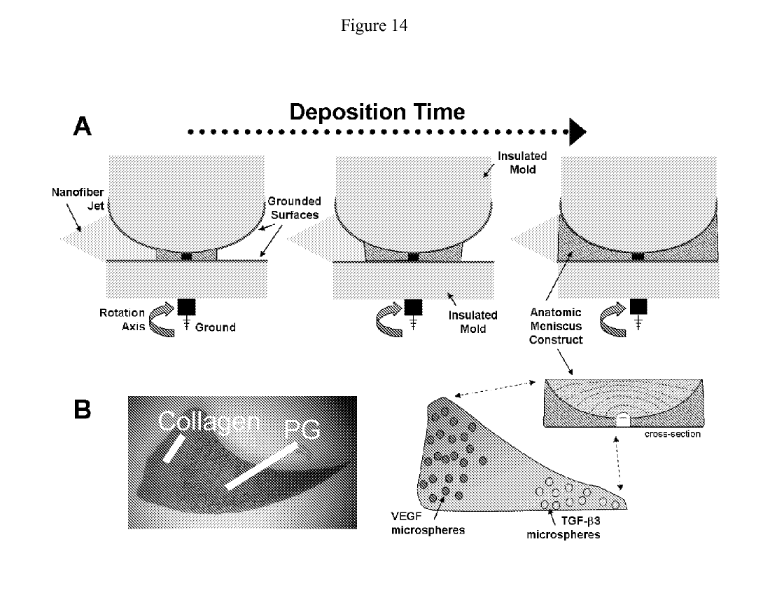

More specifically, the inner mandrel will be an aluminum shaft of about ¼″ diameter (though other dimensions may be used as dictated by the needs of the user), and the outer shaft will be about 1″ in diameter. Milled into this shaft will be a wedge shaped cleft as shown in FIG. 14; these exemplary sizes were chosen to match the average inner and outer diameter of a sheep meniscus and will vary depending on the needs of the used. The outer surfaces (not part of the cleft) may be electrically insulated with Teflon or other suitable material. By grounding the internal margins of this crevice, while insulating the outer surfaces, nanofibers are attracted to the annular cleft space. These fibers build up over time so as to give rise to an annular construct in a wedge form, which construct is sectioned to form a semi-circular meniscus segment. In a clinical situation, excess material at either horn is useful for anchoring the implant in bone tunnels or to other structures nearby to the implant.

The collecting mold is designed so that disassembly is possible, such that the entire annular construct can be removed from the mandrel. As the mandrel is rotating, fibers that collect first (in the depth of the crevice) will see a lower surface velocity than those that collect later, creating a spectrum alignment through the radial thickness of the formed construct, as is observed in the native tissue. Once fabrication methodologies are optimized, scaffolds are formed that contain multiple fiber populations that improve cell infiltration as well as growth-factor delivering microspheres positioned in the appropriate anatomic location.

Other configurations of mandrels, molds, and surfaces will be apparent to those of skill in the art. For example, the structures may be modified so as to form an implant suitable for use in the elbow, hip, or other joint.

The following are exemplary, non-limiting embodiments of the claimed invention and do not in any way limit the scope of the invention.

### EXAMPLE 1

To carry out this study, 1.94 μm fluorescent or 8.31 μm polystyrene spheres were mixed with 10% PEO in 90% ethanol, probe sonicated, and electrospun. For the larger spheres, 4 different bead concentrations were used. Three regions of interest (ROI) per slide were imaged and microspheres counted. Next, PCL and PEO (10%, containing 8.31 μm microspheres) solutions were dual-electrospun from separate spinnerets as in to form an intermingled mesh of distinct fibers. A portion of the formed scaffold was incubated in dH₂O to dissolve the PEO, and all samples were imaged using SEM. In a final set of studies, microspheres were fabricated using a water/oil/water double emulsion technique modified from Cohen+, Pharm Res, 1991 8:713. Briefly, 1 g of 75:25 PLGA (11.5 kDa, DURECT Corporation) was dissolved in 3 ml of dichloromethane. One ml of 0.5% BSA in dH2O was added and homogenized for 30 seconds. Next, 2 ml of 1% PVA solution was added and the solution homogenized. This mixture was poured into 200 ml of 0.2% PVA and stirred for 3 hours. Hardened microspheres were isolated by passing the mixture through a 70 micron nylon filter (BD Biosciences), centrifuged, washed, and lyophilized overnight. Different masses of BSA-loaded microspheres were added to 3 mL of 10% PEO in dH₂O, sonicated, and electrospun. Sections of each formed mat were dissolved in 1 N NaOH for 24 hr and BSA content was measured using a BCA assay and normalized to the scaffold mass.

### Results

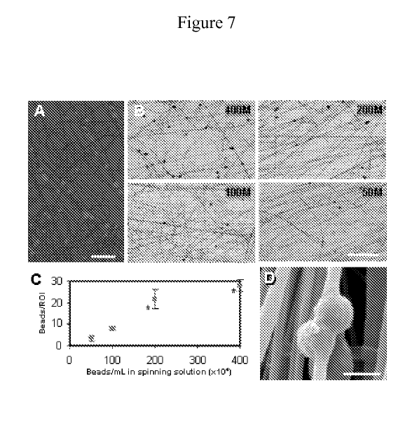

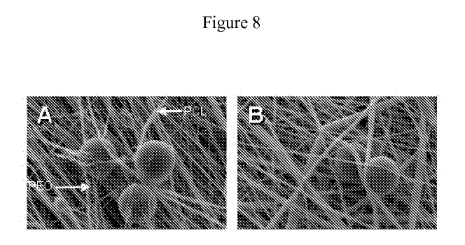

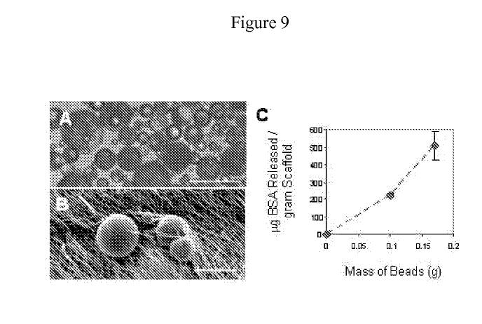

Fluorescent microspheres were successfully incorporated into electrospun PEO nanofibers (FIG. 7A). With the larger microspheres, visualization using a light microscope was possible. Doping PEO solutions with an increasing concentration of microspheres led to a dose-dependent increase in the effective bead density within the nanofiber array (FIG. 7B,C). In subsequent studies, dual electrospinning PCL and PEO/microspheres produced composite networks containing distinguishable PCL (thick) and PEO (thin, with beads) fibrous networks (FIG. 8). After aqueous incubation, PEO fibers dissolved, leaving numerous microspheres entrapped within the remaining the PCL network. To evaluate protein release, BSA-laden degradable microspheres were fabricated using the double emulsion technique (FIG. 9A). SEM confirmed that smooth spheres were created without pits or other visible irregularities (FIG. 9B). When BSA-containing microspheres were electrospun into PEO mats at increasing concentrations, an increase in BSA release was observed with NaOH-hastened degradation (FIG. 9C).

### Discussion

This example presented a novel method to incorporate biodegradable microspheres into aligned electrospun nanofibrous scaffolds. The method takes advantage of the sacrificial nature of the PEO component in our dual-component scaffolds, allowing spheres to be placed throughout the substance of the scaffold, while not interfering with the load-bearing capacity of the remaining structural fiber elements. In some embodiments, this method allows for the delivery of factors during both in vitro and in vivo repair and tissue engineering applications. For example, one might deliver a factor (or combination of factors) to promote differentiation, to improve vascular invasion, or more generally to promote matrix synthesis or other processes. By varying the concentration of microspheres within the initial polymer solution, one can readily control the density of microspheres within the network, and consequently, the quantity of biofactor released from the scaffold. This example thus demonstrates the utility of the claimed invention in dose-controlled delivery of biologically relevant compounds from a biodegradable microsphere/nanofiber network for use in fibrous tissue engineering applications.

### Example 2

To carry out this study, microspheres (MS) loaded with BSA were fabricated via the double-emulsion technique known in the art. Briefly, 1 g of 75:25 poly(lactide-coglycolide) (PLGA, inherent viscosity 0.55-0.75) was dissolved in 3.5 mL of dichloromethane. To this solution, 0.5 mL of 10% BSA was added and sonicated for 2 minutes to create a primary emulsion. Next, 2 mL of 10% PVA was added and homogenized for 1 minute. This mixture was subsequently poured into 200 mL 0.1% PVA and stirred for 3 hs. Hardened microspheres (MS) were isolated by passing the mixture through a 70 μm nylon filter, centrifuged, washed, and lyophilized overnight. To determine BSA content, 30 mg of MS were dissolved in 2 mL dichloromethane (DCM) and 1 mL of dH₂O and agitated for three hours. After phase separation, the aqueous phase was removed and BSA concentration determined via BCA assay. BSA release kinetics were determined by incubating 30 mg MS in 1 mL of PBS at 37° C. with agitation for 4 days. Each day, solutions were cleared by centrifugation and the supernatant removed and replaced with fresh PBS. BSA content in supernatants was determined as above. To determine dosing from scaffolds with bead inclusion, MS were added to 10% PEO (in dH₂O) at 0.01, 0.03, 0.05, 0.07 and 0.09 g/mL. These solutions were electrospun for 5 s onto a glass slide and imaged via light microscopy to quantify bead density. For studies with thick constructs, 5 mL of 28% poly(ε-caprolactone) in a 1:1 solution of tetrahydrofuran and N,N-dimethylformamide and 5 mL of 10% PEO in water were dual-electrospun from opposing needles onto a rotating grounded mandrel at 13 kV (10 cm) and 14 or 17 kV (9 cm) respectively. For these thicker mats, PEO solutions containing 0, 0.01, 0.05, and 0.09 g MS/mL were employed. After formation, PEO content was determined by massing samples before and after immersion in 70% ethanol overnight. MS density was visualized in scaffolds by scanning electron microscopy (SEM). BSA release was determined as above (extraction in DCM/dH₂O) and tensile testing to failure was carried out on regions of the MS-seeded scaffold with similar PEO contents. Statistics were performed by ANOVA with Tukey's posthoc tests with significance set at p<0.05.

### Results

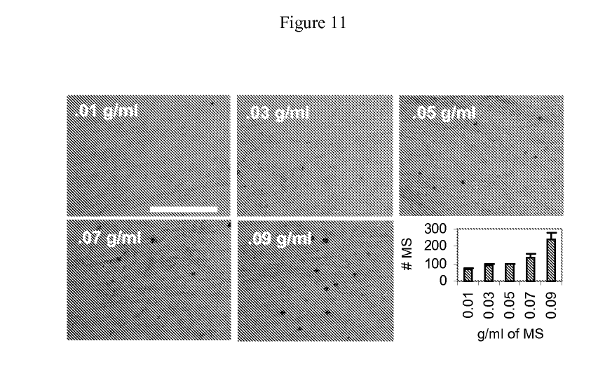

MS were successfully fabricated via the double-emulsion technique, resulting in round, hard spheres in a range of sizes. BSA encapsulation efficiency in MS was 28%±3% across 4 batches. BSA release from isolated MS was burst-like, with ˜85% released on the first day. When MS were included in PEO solutions at increasing densities and these solutions electrospun, MS were collected in fibrous networks in a dose-dependent manner. MS numerical density within the fibrous scaffold was higher (p<0.05) for solutions starting with MS densities of 0.07 and 0.09 g/mL compared to other groups (FIG. 11).

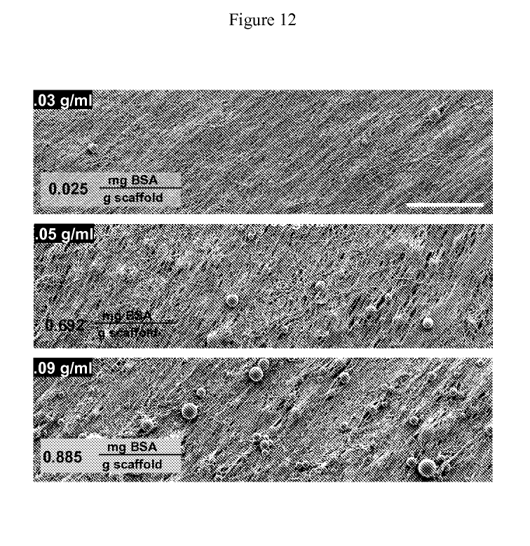

Next, PCL and PEO/MS solutions were dual electrospun onto a rotating mandrel to create an intermingled scaffold. PEO content ranged from 5-25% across the mat. Scaffolds imaged before and after hydration via SEM confirmed different MS concentrations as a function of MS density in the spinning solution (FIG. 12). BSA release was determined for samples starting with ˜10% PEO and varying bead densities in the spinning solution. BSA extraction increased with increasing MS concentration (FIG. 12, inset). Finally, samples with approximately 15% PEO were tensile tested to failure. As microsphere concentration increased, the tensile modulus (and yield stress) decreased for mats in which PEO delivered MS at 0.05 and 0.09 g/mL compared to controls (same PEO content, no MS, FIG. 13, p<0.05). However, yield strain in these scaffolds did not vary with MS inclusion at any concentration.

### Discussion

In this study, we developed a novel method for the incorporation of biodegradable microspheres in nanofibrous scaffolds. Importantly, this methods entraps microspheres within a structural PCL network in a dose-dependent manner, decoupling the required structural role of the scaffold with its potential drug-delivering capacity. Using BSA as a model protein, we demonstrated that increasing bead density increased delivery of this factor in a coordinate fashion. Increasing microsphere density was not wholly innocuous, however; mechanical properties of aligned scaffold decreased with increases in microsphere content. These data suggest that design criteria must be tailored to achieve adequate bio-factor delivery, over a sufficient duration, to exert a biologic effect, while not interfering with the structural and mechanical properties of the scaffold. In the long term, this method will allow for a range of growth factors (and growth factor combinations) that promote mitosis, vascular ingrowth or matrix secretion to be released from implanted scaffolds. Taken together, this work establishes a novel method for the incorporation of microspheres into electrospun scaffolds to generate highly functionalized scaffolds for fibrous tissue engineering.

### Example 3

In one non-limiting method for microsphere production, degradable microspheres made of poly(lactic-co-glycolic acid) (50:50, 503 H, Boehringer Ingelheim, MW 37.5 kDa) incorporating VEGF and TGF-beta 3 are prepared using a double emulsion process as described in Burdick, Biomaterials 2006; 27(3):452-9. Polymer is dissolved in methylene chloride (4 mL). Next, PBS (100 microliters) with and without 10 microg/mL VEGF (recombinant human VEGF 165, R&D Systems), TGF-beta- (recombinant human TGF-beta-3, R&D Systems), or BSA are added to the organic polymer solution, and emulsified by sonication (Vibra Cell, Sonics & Materials, Inc.). The primary emulsion is transferred to 50 mL of an aqueous 1% poly(vinyl alcohol), 0.5 M NaCl solution for a 30 sec homogenization (L4RT-A, 7500 rpm) to form a secondary emulsion. The secondary emulsion is added to an aqueous 100 mL 0.5% PVA (containing 0.5 M NaCl) solution and stirred to evaporate the organic solvent. Parameters are varied in order to obtain microspheres with a wide variety of sizes. For this application, microspheres <40 microns in diameter will be utilized via sieving through a cell strainer. Microspheres are washed, frozen with LN₂, lyophilized, and stored at −20° C.

### Example 4

### Materials and Methods

Polystyrene (PS) microspheres (MS) were from either Bangs Laboratories (diameters: 1.94 μm (fluorescent dragon green) and 8.31 μm, Fishers, IN) or Microsphere-Nanosphere (diameter: 15.7 gm, Cold Springs, N.Y.). For nanofiber formation, polyethylene oxide (PEO, 200 kDa) was from Polysciences (Warrington, Pa.) and poly(ε-caprolactone) (PCL, 80 kDa) was from Sigma-Aldrich (St. Louis Mo.). Tetrahydrofuran (THF) and N,N-dimethylformamide (DMF), used to dissolve PCL, were from Fisher Chemical (Fairlawn, N.J.). Poly lactide co-glycolide 50:50 (PLGA, inherent viscosity: 0.61 dL/g in HFIP) for microsphere fabrication was from DURECT Corp (Pelham, Ala.). Dichloromethane (microsphere fabrication) and bovine serum albumin (BSA, Cohen V fraction), chondroitin 6-sulfate sodium salt (CS), poly vinyl alcohol (PVA, 87-89% hydrolyzed), fluorescein (free acid) and rhodamine B were all from Sigma-Aldrich (Allentown, Pa.). The bicinchoninic acid (BCA) assay kit was purchased from Pierce Protein Research Products (Thermo Scienific, Rockford, Ill.). Dulbecco's phosphate-buffered saline (PBS) was purchased from Gibco (Invitrogen, Grand Island, N.Y.).

### Electrospinning Nanofibrous Scaffolds using Pre-Fabricated Microspheres

To electrospin fibers containing pre-fabricated microspheres, a high concentration of PS microspheres (1⁹-10⁹ MS/mL) was dispersed in 10% PEO in 90% ethanol or in 35.7% w/v PCL in a 1:1 mixture of THF and DMF. The suspension was sonicated for 3 minutes to disperse the MS and electrospun as in [19].

Briefly, a 10 mL syringe was filled with the electrospinning solution and fitted with a stainless steel 18G blunt-ended needle that served as a charged spinneret. A flow rate of 2.5 ml/h was maintained with a syringe pump (KDS 100, KD Scientific, Holliston, Mass.). A power supply (ES30N-5W, Gamma High Voltage Research, Inc., Ormond Beach, Fla.) applied a +15 kV potential difference between the spinneret and the grounded mandrel located at a distance of 12 cm form the spinneret. The mandrel was rotated via a belt mechanism conjoined to an AC motor (Pacesetter 34R, Bodine Electric, Chicago, Ill.). Additionally, two aluminum shields charged to +10 kV were placed perpendicular to and on either side of the mandrel to better direct the electrospun fibers towards the grounded mandrel.

### Fabrication and Electrospinning of PLGA Microsphere-Laden Nanofibrous Scaffolds

Degradable PLGA microspheres were fabricated using a double-emulsion water/oil/water technique based on [45]. Briefly, 0.5 grams of 75:25 PLGA was dissolved in 1 to 4 ml of DCM. The solution was further supplemented with 0.5 ml of 10% BSA and homogenized at high speed (setting 5) for 30 seconds using a Homogenizer 2000 (Omni International, Kennesaw Ga.). One to 2 mL of 1% PVA was then added and the entire mixture re-emulsified by homogenization for 1 minute at low speed. Hardened microspheres were collected after gentle stirring for 3 hours in 100 ml of 0.1% PVA. The collected microsphere solution was then passed through a 70 μm nylon filter (BD Biosciences, Bedford, Mass.), centrifuged, and washed 3 times in water. Fabricated microspheres were lyophilized and stored at −20° C. until use. Light microscope images were taken after fabrication, after filtration, and before lyophilization, and diameters determined using a custom MATLAB program. Microsphere density in formed nanofibers was determined after electrospinning from solutions containing 0.01, 0.03, 0.05, 0.07 and 0.09 g MS/ml PEO solution onto a glass slide for 5 seconds (n=3). For each condition, three light microscope images were obtained with similar fiber density per slide, and microspheres were counted in each image.

### Fabrication of PCL/MS Composite Nanofibrous Scaffolds

Composite nanofibrous scaffolds (PCL/PCL and PCL/PEO) containing PS microspheres (15.7 micron diameter) were formed by dual-electrospinning from two opposing spinnerets onto a common rotating mandrel as in [46]. In one configuration, a single PCL jet (2.5 mL, +15 kV, 12 cm) and a PCL jet with microspheres was spun (2.5 mL/hr, +11 to +16 kV, 6 cm), while in a second configuration, a single PCL jet was employed with the second jet containing PEO with microspheres (2 mL/hr, +16 kV, 6 cm). Microsphere densities in the spinning solutions were 0, 0.05, 0.1 and 0.2 g PS microspheres/mL electrospinning solution. After fabrication, scaffold samples were taken along the length of the scaffold, weighed, hydrated in 50% ethanol for 10 minutes, lyophilized and reweighed to determine PEO content as a function of position. Scaffolds were imaged via SEM (Philips XL20 by FEI, Hillsboro, Oreg.) before and after PEO elution to visualize MS inclusions.

### Mechanical Properties of PCL/MS Composite Nanofibrous Scaffolds

For mechanical testing, 30×5 mm strips of scaffold were excised with their long axes oriented in the fiber direction (along the circumference of the collecting mandrel). For PCL/PEO-MS scaffolds, strips containing ˜15% PEO were utilized. Before mechanical testing, all samples were soaked in 50% ethanol for 10 minutes to remove PEO, and then stored in PBS until testing. The cross-sectional area of each sample was measured using an OptoNCDT laser measuring device (Micro-Epsilon, Raleigh, N.C.) combined with a custom Matlab program. Samples were loaded into an Instron 5848 Microtester equipped with serrated vise grips and a 50 N load cell (Instron, Canton, Mass.). Strips were pre-loaded for 2 minutes to 0.5 N, after which the gauge length was noted. Samples were then preconditioned with extension to 0.5% of the gauge length at a frequency of 0.1 Hz for 10 cycles. Finally, samples were extended to failure at a rate of 0.1% of the gauge length per second. Stiffness was determined from the linear portion of the force-elongation curve, and modulus calculated by considering sample cross-sectional area and gauge length.

### Dual Release from Composite Nanofibrous Scaffolds

PLGA microspheres were formed containing two representative molecules, bovine serum albumin (BSA) to model growth factor release and chondroitin sulfate (CS) to model small molecule release. BSA-containing microspheres were prepared as above with a 10% mass/volume BSA solution encapsulated in 50:50 PLGA. CS-containing microspheres were prepared from a 20% mass/volume CS solution that was mixed with 100 μl of 1% PVA with encapsulation in 50:50 PLGA. The initial encapsulation efficiency of BSA was determined by dissolving 50 mg of fresh MS in 0.1 N NaOH containing 5% SDS with vigorous agitation for 16 hours. The supernatant was assessed via the BCA assay, with standards containing 0.1 N NaOH with 5% SDS. To determine CS encapsulation efficiency, 50 mg of MS were dissolved in 8 mL of a 1:1 solution of DCM and H₂O with vigorous agitation for 4 hours. After overnight phase separation, the aqueous phase was removed and CS content determined using the DMMB assay [48].

Long term release of CS or BSA from PLGA microspheres was evaluated via incubation in PBS (30 mg MS per 1 mL PBS) at 37° C. on a 3-D mini-rocker (Denville Scientific, South Plainfield, N.J.). At defined intervals over 5 weeks, microspheres were pelleted by centrifugation and the supernatant tested for CS content (via the DMMB assay) or BSA content (via the BCA assay) as above. At each sampling, fresh PBS was added and MS re-dispersed by gentle vortexing. Next, composites were formed to evaluate release from MS when entrapped in a PCL network. In preliminary studies, to image the composite, PCL was doped with fluorescein and PLGA microspheres were fabricated with rhodamine B. Fluorescent and light micrographs were overlaid to identify each component within the composite system. Subsequently, three microsphere-laden nanofibrous composites were constructed: one with CS-containing microspheres, one with BSA-containing microspheres, and one with a 1:1 mixture of CS- and BSA-containing microspheres. For these studies, 80 mg of scaffold cut across the length of the mandrel to ensure sample uniformity. Scaffolds were soaked in 5 ml of 50% ethanol for 10 minutes and washed in PBS to remove the PEO fraction. Scaffolds were then transferred to PBS (1 mL) and incubated as above for the MS release study. At set intervals, the supernatant was removed and CS and BSA quantified as above.

### Statistical Analyses

One-way analysis of variance (ANOVA) was carried out using GraphPad Prism software (Graphpad Software, La Jolla, Calif.) with Bonferonni's post-hoc tests (n=3 for characterization of MS density, n=5 for mechanical testing, n=5 for evalution of release kinetics), with significance set at p<0.05.

### Results

### Formation of Nanofibers with Microsphere Inclusions

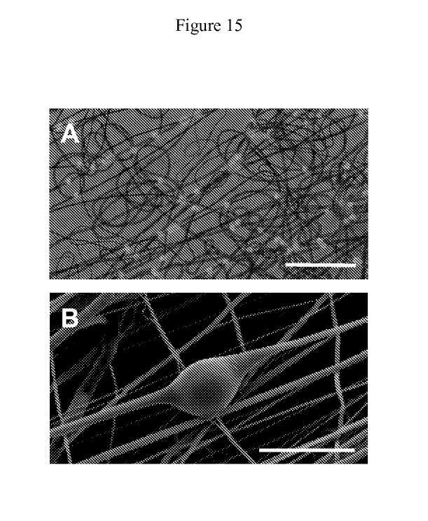

Electrospinning from a solution of PEO and pre-fabricated fluorescent polystyrene microspheres resulted in the formation of fibers with microspheres embedded along the length (FIG. 15A). Similar findings were noted when PS microspheres were electrospun from PCL solutions, with thickened regions of PCL visible around the microsphere via SEM (FIG. 15B).

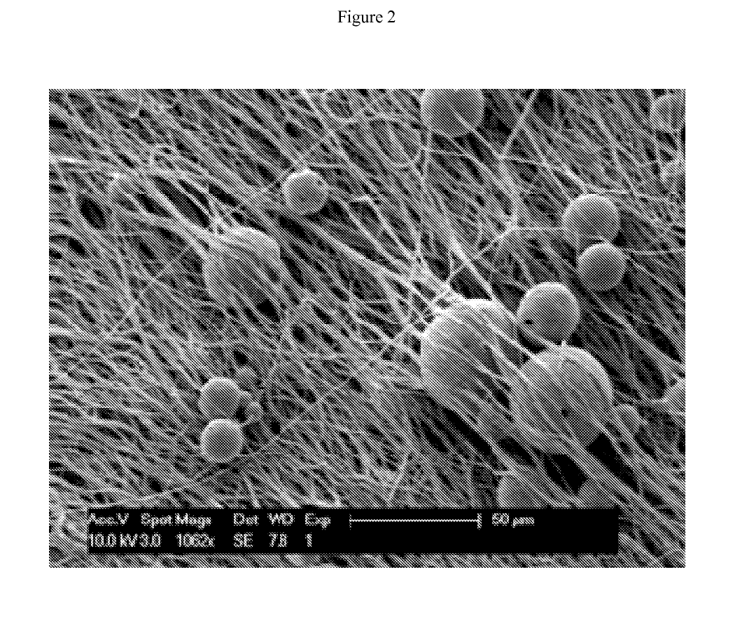

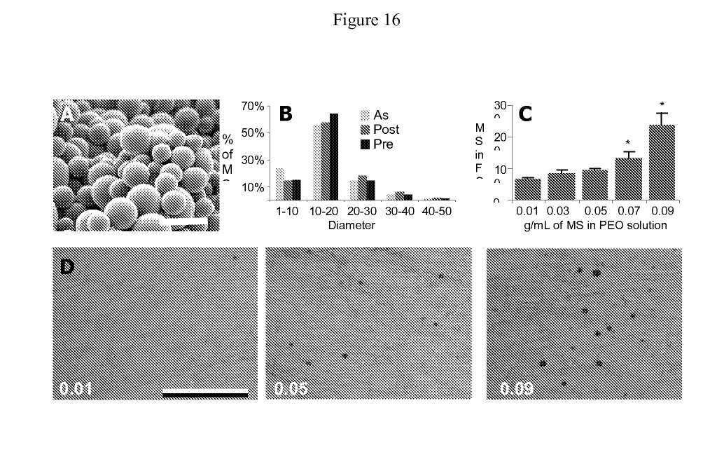

PLGA microspheres were fabricated via the water/oil/water double emulsion process (FIG. 2A). Microsphere diameters were on the order of 10-20 microns, with little change through the washing process. Increasing the density of PLGA microspheres in the PEO electrospinning solution increased the density of microspheres in the resulting fibers (FIG. 16C,D). Microsphere numerical density within the fibrous scaffold was higher for solutions starting with microspheres at 0.07 and 0.09 g/mL compared to those starting with lower microsphere concentrations (FIG. 16C, p<0.05).

### Fabrication and Electrospinning of Microsphere-Laden Nanofibrous Scaffolds

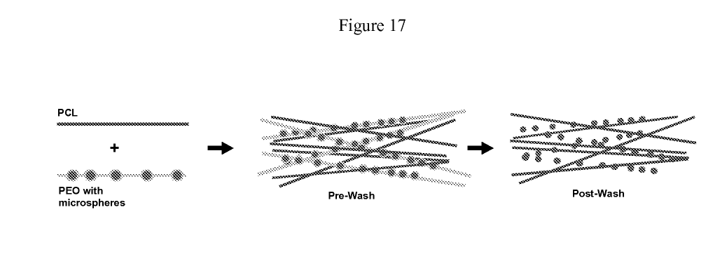

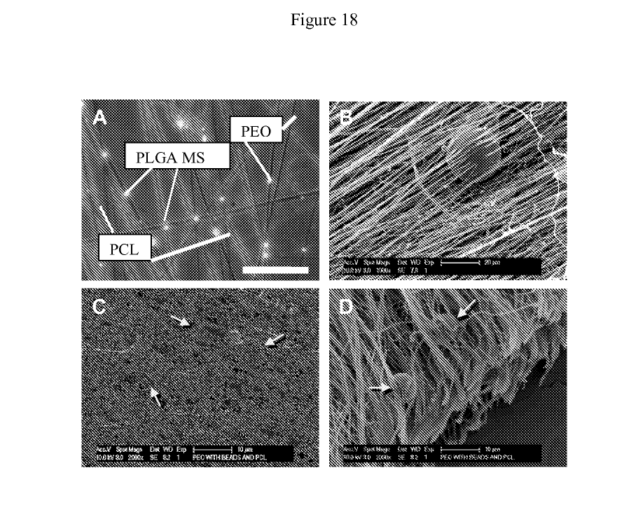

As described above, and shown schematically in FIG. 17, a novel fabrication system was developed to entrap microspheres within a fibrous scaffold. In this technique, the sacrificial PEO fiber population containing microspheres is co-electrospun with PCL onto a common rotating mandrel. Upon hydration, the sacrificial PEO fibers dissolve, resulting in continuous PCL fibers with microspheres entrapped and dispersed between. These composites were fabricated as described with fluorescent labeling of the PCL and PLGA microspheres, while the PEO component remained unlabelled (FIG. 18A; the labeled PLGA and PCL appear brighter in this grayscale figure than the unlabeled PEO). SEM images of composites before (FIG. 18B) and after (FIG. 18 C,D) hydration show that microspheres are entrapped between aligned fibers. Notably, this dispersion is seen throughout the thickness of the composite when cross sections are viewed end on (FIG. 18D).

### Mechanical Properties of Composite Scaffolds as a Function of Microsphere Inclusion

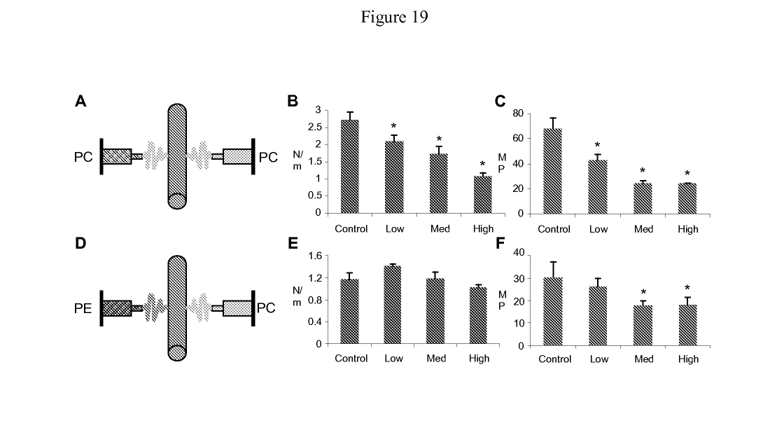

To better understand the mechanical consequences of microsphere inclusion, networks were formed in which a graded concentration of polystyrene microspheres was entrapped either within or between the nanofibers comprising the scaffold. Polystyrene MS (15.7 μm diameter) were used here as PLGA microspheres dissolve when mixed into a PCL electrospinning solution. Scaffolds were fabricated as depicted in FIGS. 19A and 19D, with one jet used to produce a pure PCL fiber population, and a second jet used to generate a fiber population of either PCL or PEO containing microspheres at increasing densities. Tensile testing showed that when microspheres were included within the PCL fiber population, both the stiffness and modulus decreased with each step of increasing microsphere density (FIGS. 19B and C). Conversely, in composites where the microspheres were entrapped between fibers after sacrificial fiber removal, no change in stiffness was observed at any microsphere density (FIG. 19E). Likewise, modulus in these composites did not differ from control values at Low microsphere densities. Due to small increases in sample thickness with increasing density of microsphere inclusion, the modulus of composites decreased at higher densities (FIG. 19F).

### Controlled Release from Microsphere-Laden Nanofibrous Composites

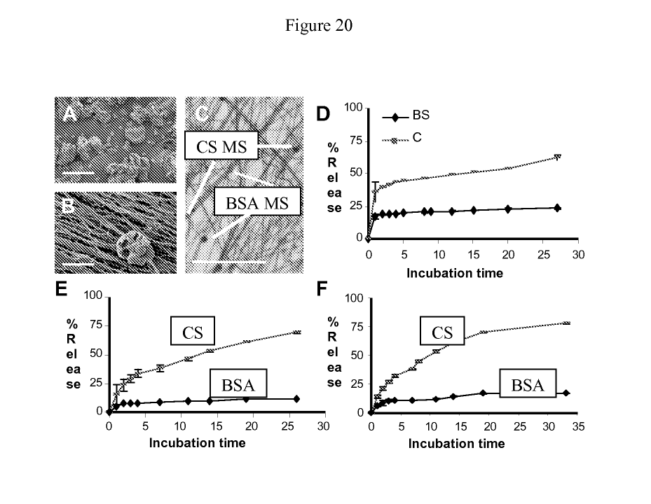

To determine whether factors could be released from the composite in a controlled fashion, BSA- and CS-containing PLGA microspheres were fabricated and release rates determined for both free microspheres and microspheres entrapped within the composite structures. The encapsulation rate for each molecule was 13% and 11%, respectively, with a burst release occurring over the first day for free microspheres, followed by a sustained release over 27 days (FIG. 20A). The initial burst release was larger from the CS-containing microspheres compared to BSA-containing microspheres. By day 27, free microspheres had degraded to the point where clumping was apparent (FIG. 20D). When one family of MS was electrospun into the composite, a more gradual release profile was observed over the first 5 days, with sustained released occurring thereafter (FIG. 20B). Contrary to naked microspheres, microspheres entrapped in nanofibrous scaffolds maintained their morphology, most likely due to physical protection and isolation when media were changed (FIG. 20E). When the MS populations were mixed 1:1 and electrospun into a single nanofibrous composite (FIGS. 20C, 20F, CS and BSA), a similar graded release profile for each molecule was observed over 35 days (FIG. 20C).

### Discussion

Electrospun nanofibrous scaffolds are a promising tool for fibrous tissue engineering as they provide excellent structural cues and can foster development of anisotropic mechanical properties similar to native tissues [19]. Indeed, we have grown constructs in vitro, under chemically defined conditions and with the addition of matrix-promoting growth factors that reach 50-100% of the tensile properties of native meniscus and annulus fibrosus [3] [12]. Simply providing a guided micropattern for tissue formation may not be enough, however, as both tissue development and regeneration occur in the context of a host of biologic factors whose timing and doses vary considerably. Moreover, upon implantation of a scaffold, our ability to control the chemical environment (i.e., the provision of pro-matrix forming growth factors in culture medium) is lost. Further functionalization of these scaffolds to enable delivery of drugs, growth factors or other chemicals would further our ability to both guide construct maturation and dictate cell behavior in vivo and in vitro.

Several recent reports have shown that micro-and nano-particles can be incorporated into electrospun nanofibers. In one early report, Lim and colleagues demonstrated that silica particles ranging in size from 100-1000 nanometers could be electrospun from a solution of polyacylimide to create a ‘bead on a string’ fiber morphology [49]. Also, Dong et al. incorporated two distinct populations of nanospheres into electrospun polyurethane fibers, suggesting the ability to multiplex delivered factors, but did not evaluate release [50]. Towards drug delivery, Melaiye et al. incorporated silver(I)-imidazole cyclophane gem-diol complexes into tecophilic polymer electrospun fibers, and demonstrated that release of this molecule from particles within the fibers could prevent microbial growth [51]. Finally, Qi et al. fabricated BSA-loaded Ca-alginate microspheres and emulsion electrospun the spheres within PLLA fibers. In this context, BSA released at a slower rate and with a lower initial burst than from free Ca-alginate microspheres [52]. While these previous studies represent an early effort to protect a molecule during fabrication and release it from a particle in a fiber, they did not address the mechanical characteristics of the system, and how the inclusion of particles within the fibers influences release kinetics.

Given the mechanical roles these scaffolds must play upon in vivo placement (where the tensile moduli of fiber reinforced tissues are on the order of 100 MPa [53]), we endeavored to create a system where microspheres could be delivered without significantly disrupting the overall scaffold mechanics. Inclusion of particles within fibers disrupts individual fiber architecture (FIG. 15B) and creates local stress concentrations, and thereby modifies the overall mechanical properties of the scaffold. Our composite system, in which particles are within the fibrous network (but not the fibers themselves), maintained the stiffness (FIG. 19E) of the PCL-based scaffolds at all microsphere densities explored. Conversely, when the same microspheres were included in the load-bearing PCL component, scaffold stiffness decreased even at low microsphere concentrations. Of note, while stiffness did not change in the composite, modulus did decrease at the medium and high microsphere concentrations. This was most likely due to a small increase in cross sectional area (decrease in fiber packing) with microsphere inclusion.

Spatial and temporal control of growth factor presentation is an important consideration in directing cell behavior during development and repair. Delivery of particles within a fiber may complicate release by coupling molecular diffusion within a fiber and/or fiber degradation with the release kinetics of the factor from the particle itself. Our approach delivers particles via a sacrificial fiber population, which is removed immediately upon hydration. When particles are of sufficient size (20 microns, in this case), they remain entrapped within the fibrous network, but are exposed directly to the aqueous environment. This approach decouples release kinetics from the microparticle from the degradation kinetics of the scaffold itself. Furthermore, using PEO allows for a compatible solvent system (water) for sacrificial fiber production, such that the PLGA microsphere structure is not disrupted with exposure to organic solvents (i.e., the DMF/THF solution used to dissolve PCL). When two model agents, BSA and CS were included in microspheres in the composite, release kinetics were independent from one another and comparable to free microspheres, suggesting that release is indeed independent of the surrounding fiber population (FIG. 20). A further interesting observation was that, when incorporated into scaffolds, microspheres maintained their spherical structure over 35 days, whereas free microspheres tended to clump together over this time scale.

The potential applications of a composite nanofibrous system that can deliver multiple factors in a controlled fashion while maintaining mechanical functionality are enumerable. For example, a cascade of growth factors (i.e., PDGF followed by VEGF) might be delivered to promote vascularization of the implanted construct [20]. This would be particularly suited for the knee meniscus, whose dense structure limits vascular regions and so limits endogenous repair. Alternatively, one might engineer the system to provide for instantaneous release of a mitogenic (i.e., FGF) or migratory factors, followed by a delayed release of a pro-matrix forming compound (i.e., TGF-beta). This construction would promote cell infiltration from surrounding tissue and division during an initial period of repair, followed by transition towards a matrix deposition phase of development.

Delivered factors also need not be solely anabolic/growth promoting. For example, microparticles might be designed to deliver proteases locally to engender local matrix disruption to enhance bridging of new matrix between the host tissue and the implanted material. Similarly, the distribution of particles need not be homogenous, with gradients of local release established both through the depth and along the fiber plane.

While the results of this study are promising, and the system meets our stated design criteria, some issues remain to be optimized. First, it is not clear how microsphere size influences mechanical properties; in this work, microspheres were on the order of 20-30 microns. Larger microsphere sizes might further disrupt mechanical properties, while smaller particles could be lost from the scaffold through the porous structure. Additional studies are required to examine these variables. Another point of optimization involves the steric and biologic influences of the particles themselves. We have previously demonstrated that both meniscus fibrochondrocytes and mesenchymal stem cells attach to and infiltrate electrospun PCL scaffolds [11, 12, 19, 46]. While the microspheres in this formulation are composed of a biocompatible material (PLGA), local pH changes with PLGA degradation might influence cellular activity. Further, sacrificial fibers were used here to deliver microspheres. We previously utilized these sacrificial fibers (at a level of ˜40-60% of the composite) to increase scaffold porosity and enhance cell infiltration into the depth of the aligned nanofibrous structure [46]. For microsphere inclusion, our highest PEO content was on the order of 15%. It remains to be determined how this low level of sacrificial fibers (and the potential decrease in fiber packing due to the microspheres themselves) influences cell infiltration. Future iterations may utilize a multiple spinneret system comprised of one source jet delivering PCL or another slow-degrading structural fiber population, one source jet delivering PEO fibers, and the final jet delivering microspheres through additional sacrificial PEO fibers. Such a multi jet system would also allow for the provision of additional mechanical functionality via variation in the mechanical properties of the PCL or slow eroding component [54]. A final point of optimization is the microspheres themselves. We used a traditional fabrication technique (water/oil/water emulsions) to entrap model compounds in order to demonstrate multi-factor release. While sufficient for proof of principle, we did observe the common burst release with each compound. Others have shown that microsphere fabrication methods can be tuned to enable release with a multitude of different profiles, including constant, early burst, and late burst [55]; such methods would be useful in further tuning towards the intended biologic applications described above.

### Conclusions

Overall, this study describes a novel approach for the creation of drug-delivering anisotropic nanofibrous scaffolds for fibrous tissue engineering. In this fabrication method the inclusion of microspheres does not significantly modify the mechanical properties of the scaffold or the release properties of the microspheres entrapped within the composite. Importantly, multiple populations of microspheres releasing unique factors can be incorporated, allowing for the complex control of cellular behavior through spatially and temporally-tuned release. Vascular recruitment, cellular phenotype and matrix elaboration may all be dictated via the proper release of single or multiple factors from these composites. Rather than simple mechanical guidance, this advanced composite provides higher order functionality for mechanical and biologic guidance of tissue regeneration.

### Additional Analysis

By employing multiple jets, the present invention provides discrete aligned fiber populations within a scaffold to form dynamic structures with the potential to improve tissue maturation. Using the disclosed methods, PCL/PEO/collagen scaffolds—along with scaffolds that include various combinations of fibers and biological materials having different mechanical and degradation profile—exhibit controllable mechanical and biologic properties that vary with differing component ratios, and that collectively these alterations will foster cell infiltration and maturation of meniscus constructs in vitro and in vivo. Without being bound to any particular theory, it is believed that collagen coatings improve cell adhesion and that pure collagen nanofibrous scaffolds are better infiltrated by cells (though much weaker mechanically) than are synthetic counterparts. Composite scaffolds that retain a synthetic backbone will retain their as-formed mechanical properties, while the PEO component will create initial porosity, and the collagen component can be remodeled with maturation via normal biologic mechanisms (i.e., the action of extracellular proteases, such as MMPs).

In addition to rapid cell colonization, vascularization and localized matrix deposition will be critical for the maturation and integration of the construct once implanted. In this proposal, we develop a novel technique for situating microspheres within the fibrous network to serve as drug delivery reservoirs. Using these spheres, controlled and localized delivery of vascular endothelial growth factor (VEGF) and transforming growth factor-beta3 (TGF-beta-3) will be investigated. It is known that VEGF is a potent recruiter of vascular endothelial cells. VEGF has recently been coated onto polymeric suture materials to improve meniscus healing after suture repair. Controlled delivery of VEGF also promotes robust vascular cell invasion and blood vessel formation in porous foams implanted subcutaneously.

This promising molecule has not been used in conjunction with aligned nanofibrous scaffolds for meniscus repair applications. TGF-β3, on the other hand, is used routinely in tissue culture for promoting fibrochondrogenesis (proteoglycan and type II collagen deposition), and is a key biologic mediator of matrix deposition in our in vitro studies. After developing this system, these locally delivered factors may be used to promote of region-specific matrix formation in vitro and regional vascular ingrowth in vivo by forming planar scaffolds with gradations in microsphere positioning.

The present invention may also be used to form anatomically-shaped scaffold constructs. As one non-limiting example, an anatomically correct meniscus shaped construct formed is suitably formed by direct electrospinning onto a molded collecting mandrel. Such constructs may then be used for implantation into subjects.

What is claimed:

1. A composition, comprising:
one or more first fibers comprising a first polymeric material,
the first polymeric material having a first rate of degradation when contacted with an fluid medium;
one or more second fibers comprising a second polymeric material,
the second polymeric material having a second rate of degradation when contacted with an fluid medium,
the second rate of degradation being faster than the first rate of degradation; and
one or more microspheres,
the one or more microspheres having a third rate of degradation when contacted with a fluid medium.

2. The composition of claim 1, wherein the second fiber degrades essentially instantaneously upon contact with an aqueous medium.

3. The composition of claim 1, wherein a microsphere comprises one or more agents.

4. The composition of claim 3, wherein an agent comprises an active agent, a label, or any combination thereof

5. The composition of claim 4, wherein a label comprises a fluorescent label, a magnetic label, a radioactive label, or any combination thereof.

6. The composition of claim 4, wherein an active agent comprises a growth factor, a pain reliever, a protein, a vitamin, a chemotherapy agent, a pharmaceutical, or any combination thereof.

7. The composition of claim 1, wherein a microsphere comprises poly(lactic-co-glycolic acid), polyanhydride, or both.

8. The composition of claim 1, wherein the third rate of degradation is slower than the second rate of degradation.

9. The composition of claim 1, wherein the first polymeric material comprises a polyester, a polyurethane, a protein, or any combination thereof

10. The composition of claim 9, wherein the polyester comprises poly(caprolactone).

11. The composition of claim 9, wherein the protein comprises silk.

12. The composition of claim 1, wherein the second polymer material comprises a polyester, poly(ethylene oxide), a protein, or any combination thereof.

13. The composition of claim 12, wherein the polyester comprises a poly-β-amino ester.

14. The composition of claim 12, wherein the protein comprises collagen.

15. The composition of claim 1, wherein one or microspheres resides at least partially within a second fiber.

16. The composition of claim 1, wherein one or microspheres resides adjacent to a first fiber, a second fiber, or both.

17. The composition of claim 1, wherein one or more first fibers are intertwined with one or more second fibers.

18. The composition of claim 1, further comprising a biological material.

19. The composition of claim 18, wherein the biological material comprises collagen.

20. The composition of claim 1, further comprising a cell.

21. The composition of claim 1, wherein a first fiber, a second fiber, or both, comprises a cross-sectional dimension of from about 1 to about 10,000 nm.

22. The composition of claim 1, wherein two or more first fibers are aligned.

23. The composition of claim 1, wherein two or more second fibers are aligned.

24. The composition of claim 1, wherein one or more first fibers are aligned with one or more second fibers.

25. The composition of claim 1, wherein essentially all of the fibers are characterized as being aligned with one another.

26. The composition of claim 1, wherein one or more first fibers are intertwined with one or more second fibers.

27. A method of fabricating an composition, comprising:
forming one or more first fibers from a first solution comprising a first polymer,
the first solution comprising one or more microspheres,
the first fibers having a first rate of degradation when contacted with a fluid medium, and
the microspheres having a third rate of degradation when contacted with a fluid medium; and
forming one or more second fibers from a second solution comprising a second polymer,
the second fibers having a second rate of degradation when contacted with a fluid medium,
the second rate of degradation being slower than the first rate of degradation.

28. The method of claim 27, wherein the forming the one or more first fibers, the one or more second fibers, or both, comprises electrospinning

29. The method of claim 27, wherein the microspheres are essentially inert to the first solution.

30. The method of claim 27, wherein a first fiber, a second fiber, or both, comprises a cross-sectional dimension of from about 1 to about 10,000 nm.

31. The method of claim 27, further comprising intertwining one or more first fibers with one or more second fibers.

32. A composition, comprising:
one or more first fibers comprising a first polymeric material,
the first polymeric material having a first rate of degradation when contacted with an fluid medium;
one or more microspheres disposed adjacent to one or more first fibers,
the one or more microspheres having a second rate of degradation when contacted with a fluid medium.

33. The composition of claim 32, where one or more microspheres are disposed between two or more first fibers.

34. The composition of claim 32, where one or more microspheres are bound to one or more first fibers.

35. The composition of claim 32, wherein the second rate of degradation is slower than the first rate of degradation.

36. The composition of claim 32, wherein the first polymeric material comprises poly(caprolactone).

37. The composition of claim 32, wherein a first fiber comprises a cross-sectional dimension of from about 1 to about 10,000 nm.

38. The composition of claim 32, wherein two or more fibers are aligned with one another.

39. A method of delivering an agent to a subject, comprising:
disposing within the subject a composition according to claim 1 or claim 32 so as to give rise to at least a portion of the composition being contacted with a fluid medium.

40. The method of claim 39, comprising placing the composition adjacent to a tendon, a ligament, a meniscus, cartilage, an annulus fibrosus, cardiac tissue, vascular tissue, neural tissue, or any combination thereof.

41. The method of claim 39, further comprising securing at least a portion of the composition to at least a portion of a tendon, a ligament, a meniscus, cartilage, an annulus fibrosus, cardiac tissue, vascular tissue, neural tissue, or any combination thereof

42. A method of delivering an agent, comprising:
contacting a composition with a fluid medium, the composition comprising
one or more first fibers comprising a first polymeric material,
the first polymeric material having a first rate of degradation when contacted with the fluid medium,
one or more second fibers comprising a second polymeric material,
the second polymeric material having a second rate of degradation when contacted with an fluid medium,
the second fibers degrading faster than the first fibers when contacted with the fluid medium, and
one or more microspheres disposed among the first and second fibers,
the one or more microspheres degrading more slowly than the second fibers when contacted with the fluid medium, and
the one or more microspheres being capable of releasing one or more agents when contacted with the fluid medium;
the contacting being performed so as to at least partially degrade one or more second fibers,
the contacting being performed such that one or more microspheres remains disposed among at least the first fibers, and
the contacting being performed such that one or more microspheres releases one or more agents into the environment exterior to the microsphere.
